(19) United States
(12) Patent Application Publication
(10) Pub. No.: US 2006/0210623 A1
(43) Pub. Date: Sep. 21, 2006

(54) Sustained release delivery of isradipine

(76) Inventors: Paul E. Stach, Tampa, FL (US); Balaji V. Kadri, Tampa, FL (US); George Bobotas, Tarpon Springs, FL (US); Abdel A. Fawzy, Easton, PA (US)

Correspondence Address: ARENT FOX PLLC, 1050 CONNECTICUT AVENUE, N.W., SUITE 400, WASHINGTON, DC, 20036, US

(21) Appl. No.: 11/377,648

(22) Filed: Mar. 17, 2006

Related U.S. Application Data
(60) Provisional application No. 60/662,370, filed on Mar. 17, 2005.

(51) Int. Cl. A61K 9/52; A61K 9/22

(52) U.S. Cl. 424/457; 424/468

(57) ABSTRACT

Sustained release oral formulations of israpidine, methods of preparing same, and methods of using sustained release oral formulations to provide controlled delivery of israpidine. The sustained release oral formulations include israpidine and a sustained release polymer, and provide substantially zero order release of israpidine over an extended period.

### FIELD OF THE INVENTION

The present invention relates to compositions and methods of treating human subjects with a sustained release delivery of isradipine.

### BACKGROUND OF THE INVENTION

Isradipine is a potent dihydropyridine calcium antagonist with a higher affinity for calcium channels in arterial smooth muscles than those in the myocardium. Essentially, isradipine interferes with the movement of calcium into heart muscle cells and the smooth muscle cells in the walls of the arteries. Isradipine relaxes, i.e., widens, blood vessels so the heart does not have to pump as hard. As a result, isradipine is typically used in the treatment of ischemic heart disease and systemic hypertension.

Generally, an extended (also known herein as “sustained”) release dosage form of isradipine is more desirable than an immediate-release dosage form. Ideally, the former form may provide patients with a convenient dosage regimen that allows less frequent dosing, thus enhancing compliance. Extended release dosing may also reduce peak-related side effects, maintain therapeutic concentrations throughout the dosing period avoiding periods of insufficient therapeutic plasma concentrations between doses.

Within the spectrum of the extended release dosage forms, certain types are more preferred than others. Drugs having first-order kinetics exhibit an initial high-blood level of the drug followed by an exponential decrease in blood concentration. It has been noted that this kinetic model may be problematic because therapeutic effectiveness will not ensue when blood concentrations of the drug fall below certain levels. Furthermore, some drugs are toxic at high-blood level concentrations, and it is difficult to achieve a balance between effective levels and toxic levels when blood concentrations fall off so rapidly.

A more ideal delivery of drugs would follow zero-order kinetics, wherein blood levels of drugs would remain constant throughout the delivery period. This ideal delivery is particularly important in certain classes of medicines intended, for example, for antibiotic delivery, heart and blood pressure maintenance, pain control, and antidepressants. See, Landgraf et al, Polymer Microcarriers Exhibiting Zero-Order Release, Drug Delivery Technology, http://www.druqdeliverytech.com/cqi-bin/articles.cqi?idArticle=114 (2005). Accordingly, extended release dosages generally prefer a zero order release profile. In a typical zero order profile, the release of active ingredient would be relatively constant over reasonably extended periods of time.

A zero order release profile is desired with respect to isradipine's beneficial heart effects. There is an unmet need in the art for sustained release tablet and capsule delivery systems to administer a pharmaceutically effective amount of isradipine to subjects in a zero-order manner or in a substantially zero-order manner.

### SUMMARY OF THE INVENTION

The present invention overcomes the above-mentioned problems, as well as others, by providing a delivery system and method to administer isradipine to subjects over a steady rate over a prolonged period of time. One aspect of the present invention discloses a system and method to deliver a therapeutically effective amount of sustained release isradipine to a human subject to treat heart related disorders, including hypertension and ischemia. The delivery of isradipine has a zero order kinetic profile or substantially a zero order kinetic profile. In some embodiments, the present invention is directed to delivering a therapeutically effective amount of sustained release isradipine in an oral dosage form, such as a tablet or capsule, once to twice daily.

In some embodiments, the present invention is directed to delivery system of sustained release isradipine, wherein a therapeutically effective amount of isradipine and at least one pharmaceutically effective excipient are contained in a delivery system. In one embodiment, the delivery system comprises a tablet or core of isradipine and excipients, coated with an outer coating including a sustained release polymer. In another embodiment, the delivery system comprises an inert core that is coated with israpidine, and then further coated with an outer coating including a sustained release polymer. In a further embodiment, the delivery system comprises a matrix tablet or core of israpidine and a sustained release polymer. The various embodiments of the present invention facilitate sustained release of israpidine, in accordance with a zero-order or a substantially zero-order kinetic profile.

In additional embodiments, isradipine is combined with a filler and a polymer in a core, which may optionally be coated. In one variation, the polymer is a polymerized acrylate, such as Eudragit® RS, which is typically used as a coating, but in this case is used in the core. The polymer is preferably present from about 12% to about 30% by weight of the core. In another variation, the filler is preferably lactose, e.g., in the form of Fast Flo®, in an amount from about 60% to about 80% by weight of the core. Optional excipients are added as necessary to facilitate the administration and/or manufacturing of the oral dosage form.

In yet other embodiments, the present invention is directed to delivery systems for sustained release isradipine, wherein a therapeutically effective amount of isradipine in a substantially zero-order sustained release delivery system releases about 0.5% or more of isradipine at 0.5 hours, about 10-40% isradipine at 6 hours, about 30-65% isradipine at 12 hours. According to additional embodiments, about 60-90% isradipine is released at 18 hours, and at least about 95% israpidine is released at 24 hours. The dissolution specifications were obtained under the following conditions:

| | |
|---|---|
| Medium: | 0.2% DDAO |
| Volume: | 500 mL |
| Apparatus: | USP Apparatus 2 (paddles) |
| Speed: | 50 rpm |
| Temperature: | 37° C. +/− 0.5° C. |
| Sample Volume: | 1.5 mL |

Any of the embodiments illustrated above and below stand independently or features may be combined to achieve preferred embodiments. Additional advantages and novel features of the invention will also become more apparent to those skilled in the art upon examination of the following or upon learning by practice of the invention.

### BRIEF DESCRIPTION OF THE DRAWINGS

FIG. 1 is a graph depicting the data presented in Table 6 of the percent of isradipine released over a prolonged period of time in exemplary formulations having various punch size and amount of isradipine, as compared to a prior art formulation, in accordance with some embodiments of the present invention;

FIG. 2 is a graph depicting the data presented in Table 6 of the dose percent of isradipine released over a prolonged period of time in exemplary formulations having various amounts (5, 6, 12, 18 mg/tablet) of isradipine, as compared to the prior art formulation, in accordance with some embodiments of the present invention;

FIG. 3 is a graph depicting the data presented in Table 6 of the percent of isradipine released over a prolonged period of time in exemplary formulations having various punch sizes and various amounts of isradipine, as compared to the prior art formulation in the prior art, in accordance with some embodiments of the present invention;

FIG. 4 is a graph depicting the data presented in Table 6 of the dose amount of isradipine released over a prolonged period of time in two exemplary formulations, in accordance with some embodiments of the present invention;

FIG. 5 is a graph depicting the data presented in Table 6 of the dose percent of isradipine released over a prolonged period of time in ¼ inch punch exemplary formulations having various amounts of isradipine, as compared to the prior art formulation, in accordance with some embodiments of the present invention;

FIG. 6 is a graph depicting the data presented in Table 6 of the dose percent of isradipine released over a prolonged period of time in exemplary formulations having 6 mg/tablet of isradipine having various hardness values, as compared to the prior art formulation and a 5 mg/tablet sustained release formulation of the present invention, in accordance with some embodiments of the present invention;

FIG. 7 is a graph depicting the data presented in Table 6 of the dose percent of isradipine released over a prolonged period of time in exemplary formulations having 12 mg/tablet of isradipine having various punch sizes, as compared to the prior art formulation and a 5 mg/tablet sustained release formulation of the present invention, in accordance with some embodiments of the present invention;

FIG. 8 is a graph depicting the data presented in Table 6 of the dose percent of isradipine released over a prolonged period of time in exemplary formulations having 12 mg/tablet of isradipine having various hardness values, as compared to the prior art formulation and a 5 mg/tablet sustained release formulation of the present invention, in accordance with some embodiments of the present invention;

FIG. 9 is a graph depicting the data presented in Table 6 of the dose percent of isradipine released over a prolonged period of time in exemplary formulations having 18 mg/tablet of isradipine, as compared to the prior art formulation and a 5 mg/tablet sustained release formulation of the present invention, in accordance with some embodiments of the present invention;

FIG. 10 is a graph depicting the data presented in Table 6 of the dose percent of isradipine released over a prolonged period of time in exemplary formulations having 18 mg/tablet of isradipine having various hardness values, as compared to the prior art formulation and a 5 mg/tablet sustained release formulation of the present invention, in accordance with some embodiments of the present invention;

Other features of the present invention will become apparent from the following detailed description considered in connection with the accompanying drawings, which disclose multiple embodiments of the present invention. It should be understood, however, that the figures are designed for the purpose of illustration only and not as a definition of the limits of the invention. Additional advantages and novel features of the invention will also become apparent to those skilled in the art upon examination of the following or upon learning by practice of the invention.

### DESCRIPTION OF THE PREFERRED EMBODIMENTS

The present invention discloses a system and method to deliver sustained (i.e., extended) release isradipine to a human subject to treat heart related disorders, including hypertension and ischemia. The system and method of the present invention deliver isradipine in a constant manner, wherein the isradipine has a kinetic profile more akin to a zero order profile than the kinetic profile of the prior art. Accordingly, the sustained release isradipine of the present invention provides a more steady and constant release of isradipine in a subject than conventional sustained release isradipine.

The present invention has been formulated to provide a substantially zero-order release, or preferably a zero-order release. Thus, the duration of release is related to the amount of isradipine in the delivery system, i.e., the isradipine is released from a tablet or capsule at a substantially constant rate until the isradipine is substantially dissipated. In the zero-order reaction, the timed duration of the release of isradipine is independent (or substantially independent) of the concentration of isradipine in the delivery system. For the purposes herein, “substantially zero-order” means within 20% of zero-order, and preferably within 10% of zero-order, for at least about six continuous hours while the israpidine is being delivered, preferably for at least about eight continuous hours, more preferably for at least about ten continuous hours, and most preferably for at least about twelve continuous hours.

The delivery system of the present invention includes a therapeutically effective amount of isradipine, combined with one or more pharmaceutically acceptable excipients effective to provide sustained release.

Generally, excipients are inert or slightly active substances used in preparing delivery systems as a vehicle or medium of administration for the medicinal agents. Excipients, for example, serve to solubilize, suspend, thicken, dilute, emulsify, stabilize, preserve, protect, color, and fashion the active ingredients into an applicable and efficacious preparation that is safe, convenient, and otherwise acceptable for use. Moreover, excipients are in a suitable amount such that the excipients do not deleteriously affect the delivery of isradipine.

In the present invention, excipients and isradipine are combined in a sustained release formulation. Two types of sustained release formulation are particularly preferred, one in which a sustained released polymer comprises a coating, and the other in which a sustained release polymer comprises a matrix core. However, other sustained release formulations that comprise israpidine and excipients are also envisioned in accordance with the present invention.

1. Coated Sustained Release Formulations

Generally, the coated sustained release formulations of the present invention include two or more components. The first component is a tablet or core, which can contain the active agent therein (i.e., isradipine), or can be an inert carrier coated with a coating that contains the active agent, optionally in association with conventional excipients. The second component is a sustained release coating, formed from a sustained release polymer, which envelops or substantially envelops the tablet or core. This sustained release coating is responsible for giving the active agent its particular controlled and/or delayed release characteristics. In this variation, the outer coating dissipates upon reaching the gastrointestinal tract, thereby allowing water to enter the tablet through the semipermeable membrane formed as the outer coating dissipates.

The tablet or core may be prepared by a number of techniques known in the art. Typically, the active agent may be combined with an inert carrier material and then formed into spheres, beads, tablets, etc. Alternatively, an inert bead, typically a starch or sugar sphere, may be coated with the active agent using any of the conventional coating technologies used in the pharmaceutical arts. Sugar spheres are preferred, but any pharmaceutically-acceptable inert carrier may be utilized in this alternative embodiment.

The sustained release polymer outer coating is responsible for giving the active agent its particular sustained and controlled release characteristics. The coating may be produced, for example, from polymerized acrylates or copolymers of acrylic acid and methacrylic acid or esters of either monomer (hereinafter polymerized acrylates). The sustained release polymer of the delivery systems of the present invention may also be prepared from one of the organosiloxane oral coating materials known in the art such as polydimethylsiloxane, polydiethylsiloxane, etc.

Polymerized acrylates as well as copolymers of acrylic acid and methacrylic acid or esters of either monomer are known in the art and are available from many commercial sources. Examples of such copolymers include poly(methyl methacrylate), poly(ethyl methacrylate), poly(butyl methacrylate), poly(isobutyl methacrylate), poly(isobutyl methacrylate), poly(phenyl methacrylate) etc. The polymeric coating may optionally contain a sufficient quantity of a suitable plasticizer. Examples of such plasticizers include acetyl triethyl citrate, dibutyl phthalate, tributyl citrate, triethyl citrate, acetyl tributyl citrate, propylene glycol, triacetin, polyethylene glycol and diethyl phthalate.

The sustained release polymer may also be selected from a variety of materials that are typically utilized in the pharmaceutical arts. These may include any of a variety of water insoluble polymers, such as, for example, ethylcellulose, carboxy methoylcellulose (CMC), hydroxypropyl methylcellulose (HPMC), polyvinylpyrrolidone, cellulose acetate, cellulose propionate, cellulose acetate butyrate, polyethylene, polypropylene, polyethylene oxide, polyvinyl acetate, polyvinyl chloride, etc. A minor proportion of a water-soluble polymer may also be included in the polymeric coating. Examples of such polymers include methylcellulose, hydroxypropyl cellulose, polyethylene glycol, etc. These coatings may also include conventional excipients such as plasticizers, antifoaming agents, antiadherants, etc.

The outer coating comprising a sustained release polymer may be applied to the tablet or core using methods and techniques known in the art. Typically a suspension, emulsion, or solution of the polymeric coating is prepared as would be known in the art. The amount of fluidized polymeric coating required in the coating process may be readily calculated depending upon the amount of sustained release polymer desired. The fluid sustained release polymer may be applied to the central tablet or core by a number of coating techniques known in the art. Examples of suitable coating devices include fluid bed coaters, pan coaters, etc.

A preferred acrylic polymer that can be used as the outer coating technology for sustained release delivery systems is Eudragit® (manufactured by Röhm GmbH & Co. KG, Darmstadt, Germany).

A preferred form of Eudragit® is Eudragit® RS. Eudragit® RS is a water-insoluble, swellable film former based on neutral methacrylic acid esters with a small proportion of trimethylammonioethyl methacrylate chloride, and the molar ratio of the quaternary ammonium groups to neutral ester groups is 1:40 (corresponding to roughly 25 meq./100 g). Eudragit® RS is freely miscible, and typically pore-free films are obtained at a thickness of about 10 to 30 μm. Drug diffusion then occurs through swellable, hydrophilic regions formed in the polymer matrix by quaternary ammonium groups. The quaternary ammonium groups, present as chlorides in Eudragit® RS polymers are completely dissociated in the physiological pH range of about 1 to 8. In the initially formed film, the permeability of the coating is therefore independent of pH.

In order to obtain films of adequate flexibility, about 20% plasticizer or polymer is typically added to the dispersions of Eudragit® RS. Glidants, like talc, micronized amorphous silica gel, and glycerol monostearate, facilitate spray application to the tablets or cores.

Typically, in Eudragit® RS, polymer films form diffusion cells. High concentrations of active ingredient in the tablets or cores often leads to the formation of saturated solutions in these cells. This allows a constant amount of active ingredient to be released per unit of time.

A particularly preferred coating polymer is an acrylate polymer with a low permeability, such as Eudragit® RSPO, which is a pH independent polymer powder. It should be understood by those skilled in the art that other polymers having properties similar to Eudragit® RS and Eudragit® RSPO can be used, and freely substituted in the present invention.

Some embodiments of the present invention include delivery systems such as coated tablets and capsules containing coated cores, where the delivery systems contain from about to 2.5 mg to about 25 mg of isradipine. Preferably, the amount is from about 5 mg to about 20 mg, and more preferably the amount is between from about 6 mg to about 18 mg. In some variations, the dosage amount of isradipine is 5 mg, 6 mg, 12 mg, and/or 18 mg in one tablet or capsule. A 10 mg amount of isradipine, as well as other amounts within the range provided above, are contemplated by the present invention.

Generally, the isradipine content of the sustained release dosage forms of the present invention is proportional to the amount of excipients. Accordingly, the percentage of excipients and isradipine generally remains the same in each dosage amount of isradipine. Embodiments of the present invention are also provided that relate to the ratio of israpidine to excipients. In some embodiments, the ratio of isradipine to excipients ranges from about 5:1 to about 1:5. Preferred embodiments have ratios from about 4:1 to about 1:4. More preferred embodiments have a ratio of about 2:7.

2. Sustained Release Matrix Formulations

The sustained-release forms of administration, according to the present invention, can also contain and deliver isradipine in a sustained-release matrix, preferably as a uniform distribution. In accordance with this embodiment, the active agent (i.e., israpidine) is combined with a sustained release polymer to form a sustained release matrix of the active agent. Such matrices may be provided as beads for use in capsules, or as compressed tablets.

Matrix materials that can be used are typically physiologically compatible, hydrophilic materials known to those skilled in the art. The hydrophilic matrix materials used are preferably polymers and particularly preferably cellulose ethers, cellulose esters and/or acrylic resins. The matrix materials used are very particularly preferably ethyl cellulose, hydroxypropyl methyl cellulose, hydroxypropyl cellulose, hydroxymethyl cellulose, poly(meth)acrylic acid and/or derivatives thereof such as their salts, amides or esters.

Other preferred matrix materials are those consisting of hydrophobic materials such as hydrophobic polymers, waxes, fats, long-chain fatty acids, fatty alcohols or corresponding esters or ethers, or mixtures thereof. The hydrophobic materials used are particularly preferably C₁₂-C₃₀ fatty acid mono- or diglycerides and/or C₁₂-C₃₀ fatty alcohols and/or waxes, or mixtures thereof. It is also possible to use mixtures of the above-mentioned hydrophilic and hydrophobic materials as the sustained-release matrix material. The sustained-release matrix can be prepared by the conventional methods known to those skilled in the art.

One aspect of the present invention is directed to using a polymeric material typically employed as a coating, as described above, as the sustained release matrix material. Accordingly, embodiments of the present invention can include the “coating” polymers listed above being used as matrix material. Moreover, additional embodiments of the matrix material of the present invention can include an acrylate polymer, such as Eudragit®, preferably Eudragit® RS, and more preferably Eudragit® RSPO, or like polymers.

In some embodiments the matrix may further include one or more pharmaceutically-acceptable excipients. These excipients may include a filler and/or disintegrant, such as lactose, glucose and sucrose, starches e.g., corn starch and potato starch, cellulose and cellulose derivatives (e.g., sodium carboxymethyl cellulose, ethyl cellulose, microcrystalline cellulose, and cellulose acetate), powdered tragacanth, malt, gelatin, and talc. The filler is preferably water-soluble. One preferred filler is 316 Fast Flo® (manufactured by Foremost Farms, Baraboo, Wis.). The use of fillers, such as lactose, is generally known in the pharmaceutical arts, and as would be understood to one skilled in the art, any suitable filler may be used in conjunction with the present invention and embodiments thereof.

Optional excipients include manufacturing or production process-facilitating excipients, including lubricants, such as sodium lauryl sulfate and magnesium stearate, white wax, castor oil, palmitic acid, stearic acid, mineral oil, polyethyleneglycol, and silicon dioxides, such as Cab-O-Sil® (manufactured by Eager Plastics, Chicago, Ill.), which is a synthetic, amorphous, untreated fumed silicon dioxide. Another optional excipient may include coloring agents, such as Opadry® (manufactured by Colorcon, West Point, Pa.), which is generally used in the coating composition, or other suitable film coating system.

It is understood by one in the art that the excipients enumerated herein are provided for exemplary purposes. Excipients may be substituted, replaced or otherwise modified, by and with, suitable substances known to one skilled in the art.

Some embodiments of the present invention include matrix tablets and/or capsules containing an amount from about to 2.5 mg to about 25 mg of isradipine. Preferably, the amount is from about 5 mg to about 20 mg, and more preferably the amount is between from about 6 mg to about 18 mg. In some variations, the dosage amount of isradipine is 5 mg, 6 mg, 12 mg, and/or 18 mg in one tablet or capsule. A 10 mg amount of isradipine, as well as other amounts within the range provided above, are contemplated by the present invention.

Generally, the isradipine content of the sustained release matrix dosage forms of the present invention is proportional to the amount of excipients. Accordingly, the percentage of excipients and isradipine generally remains the same in each dosage amount of isradipine. Embodiments of the present invention are also provided that relate to the ratio of israpidine to excipients. In some embodiments, the ratio of isradipine to excipients ranges from about 5:1 to about 1:5. Preferred embodiments have ratios from about 4:1 to about 1:4. More preferred embodiments have a ratio of about 2:7.

The primary excipients, the sustained release polymer and the filler, comprise the majority of the delivery system in most embodiments. In some embodiments, the sustained release polymer (e.g., Eudragit® RSPO) is present from about 12% to about 30% by weight of core, preferably from about 14 to about 24%, and most preferably from about 20% to about 22%. In certain variations, the sustained release polymer is present at about 16%, 20%, 22%, and/or 24% by weight of core.

The filler (e.g., 316 Fast Flo®) is generally present in an amount from about 60% to about 80% by weight of core, preferably from about 65% to about 75%, and more preferably from about 69% to about 72%.

Each of the optional matrix excipients are generally present in an amount not more than 5%, preferably not more than 3%, and more preferably not more than 2%, of the total amount of the matrix core. For example, sodium lauryl sulfate may present in the amount from about 0.1% to about 0.3%, preferably about 0.2%. In another example, magnesium stearate can be present in the amount from about 0.3% to about 0.6%, preferably from about 0.4% to about 0.5%. Silicon dioxides (e.g., Cab-O-Sil®) are generally present in the amount from about 0.3% to about 0.5%, preferably from about 0.4% to about 0.5%.

3. Methods of Treatment

Some embodiments of the present invention are directed to novel methods for administering a therapeutically effective amount of isradipine. As used herein, a therapeutically “effective amount” means an amount of isradipine or pharmaceutically acceptable form thereof that is nontoxic but sufficient to provide the desired systemic effect and performance at a reasonable benefit/risk ratio attending any medical treatment.

In one variation, the isradipine is administered orally in the form of a tablet or capsule. Tablet and capsule formulations for isradipine are generally known in the art. U.S. Pat. No. 5,030,456, U.S. Pat. No. 4,783,337, U.S. Pat. No. 4,950,486, U.S. Pat. No. 4,946,687, U.S. Pat. No. 4,816,263, and U.S. Pat. No. 4,466,972 are incorporated herein in their entireties.

Other tablet and capsule formulations are also generally known in the art. U.S. Pat. No. 5,439,687, U.S. Pat. No. 5,637,309, U.S. Pat. No. 5,156,850, U.S. Pat. No. 5,208,037, U.S. Pat. No. 6,096,339, U.S. Pat. No. 6,146,662, U.S. Pat. No. 6,814,979, U.S. Pat. No. 6,773,721, U.S. Pat. No. 6,645,528, U.S. Pat. No. 5,869,097, U.S. Patent Publication No. 20040081693, U.S. Patent Publication No. 20040005359, U.S. Patent Publication No. 20020114838, U.S. Patent Publication No. 20020044962, and U.S. Patent Publication No. 20020044960 are incorporated herein in their entireties.

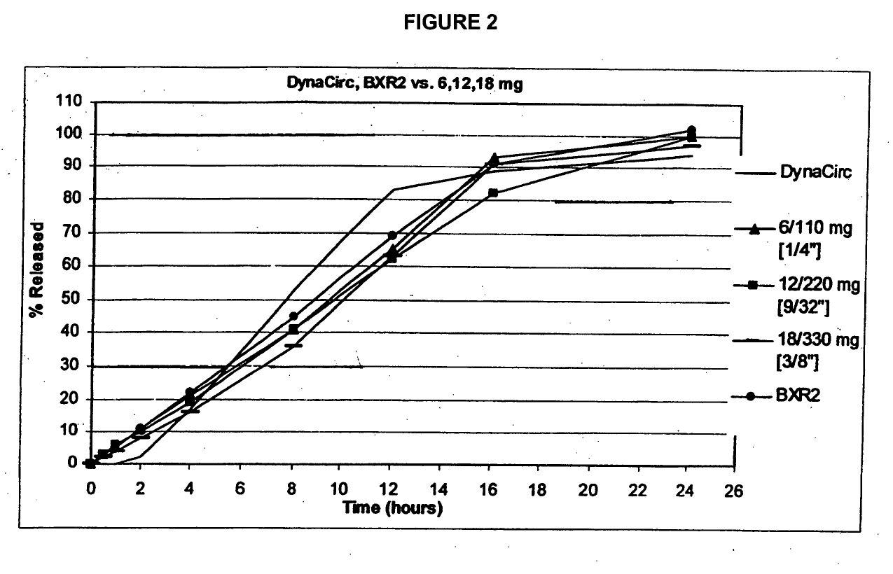

The physical size of the tablet of the present invention varies. The punch size ranges from about ¼ to about ⅜ inches. It should be understood to one skilled in the art that the dimensions of the tablet can be dictated by the amount of isradipine and excipients, and by considerations such as ease of oral administration. Some variations of the punch include ¼ inches, 9/32 inches, 5/16 inches, and ⅜ inches. The size of the punch is not dependent on the amount of isradipine contained in the tablet. In some embodiments, the hardness (kp) of the tablets ranges from 9 to 16.5. In some embodiments, the surface area ranges from 98 to 230 mm², while the volume ranges from 75 to 265 mm³. In some embodiments, the surface area to strength ratio ranges from 9.5 to 20, the surface area to volume ratio ranges from 0.85 to 1.35, and the volume to strength ratio ranges from 7 to 17. FIG. 2 provides tabulated data for exemplary formulations of the present invention.

In some embodiments of the present invention, the dosage of isradipine is taken one to two times per day. The frequency of administration, in some embodiments of the present invention, can be dictated by the plasma level of isradipine in the subject. For example, if the plasma level of isradipine is intended to remain steady, then the frequency of adminsitration should be accordingly maintained to accomplish such a goal.

In other embodiments, the sustained release tablet of isradipine of the present invention is provided once per day because it provides a constant amount of isradipine to the subject over an extended period, such as at least a twelve hour period, at least an eighteen hour period, at least a twenty hour period, at least a twenty-two hour period, or at least a twenty-four hour period. In other embodiments the extended period is a day. Accordingly, it is preferred that the isradipine is delivered at relatively the same time during the treatment period. As is understood by one skilled in the art, individual and medical factors also dictate the frequency of administration.

### EXAMPLES

Particularly preferred embodiments of the present invention will now be described with respect to the following non-limiting examples.

1. Sustained Release Tablet Formulations

1.1. 5 mg Sustained Release Israpidine Tablet

In certain 5 mg/tablet embodiments of the present invention, a 5 mg/tablet dose of isradipine is combined with a sustained release polymer (e.g., Eudragit® RSPO) from about 12 mg/tablet to about 28 mg/tablet, preferably from about 16 mg/tablet to about 24 mg/tablet, and most preferably about 20 mg/tablet. A filler (e.g., 316 Fast Flo®) is present in an amount from about 60 mg/tablet to about 80 mg/tablet, preferably from about 65 mg/tablet to about 75 mg/tablet, and more preferably from about 67.5 mg/tablet to about 72.5 mg/tablet. Sodium lauryl sulfate is optionally present in the amount from about 0.1 mg/tablet to about 0.3 mg/tablet, preferably from about 0.15 mg/tablet to about 0.23 mg/tablet, and more preferably about 0.2 mg/tablet. Optionally, magnesium stearate is present in the amount from about 0.475 mg/tablet to about 0.525 mg/tablet, preferably from about 0.49 mg/tablet to about 0.51 mg/tablet, and more preferably about 0.5 mg/tablet. Optionally, Cab-O-Sil® M5P is present in the amount from about 0.475 mg/tablet to about 0.525 mg/tablet, preferably from about 0.49 mg/tablet to about 0.51 mg/tablet, and more preferably about 0.5 mg mg/tablet. Opadry® is optionally present in the amount from about 4.8 mg/tablet to about 6 mg/tablet, preferably from about 5.2 mg/tablet to about 5.6 mg/tablet, and more preferably about 5.4 mg mg/tablet. Exemplary formulations for a 5 mg tablet of sustained release isradipine are set forth below in Table 1.

TABLE 1

| | Strength (mg) | | | | | | | | | | | |
|---|---|---|---|---|---|---|---|---|---|---|---|---|
| | 5 | | 5 | | 5 | | 5 | | 5 | | 5 | |
| Ingredient | % | mg/tab | % | mg/tab | % | mg/tab | % | mg/tab | % | mg/tab | % | mg/tab |
| Isradipine, USP | 5.0 | 5.0 | 5.0 | 5.0 | 5.0 | 5.0 | 5.6 | 5.04 | 5.6 | 5.0 | 4.0 | 4.0 |
| Eudragit RS PO | 20.0 | 20.0 | 22.0 | 22.0 | 24.0 | 24.0 | 16.0 | 14.4 | 22.0 | 19.8 | 22.0 | 22.0 |
| Modified, 316 Fast Flo | 74.0 | 74.0 | 72.0 | 72.0 | 70.0 | 70.0 | 77.4 | 69.66 | 71.2 | 64.12 | 73.0 | 73.0 |
| Sodium Lauryl Sulfate, NF | | | | | | | | | 0.2 | 0.18 | | |
| Magnesium Stearate, NF | 0.5 | 0.5 | 0.5 | 0.5 | 0.5 | 0.5 | 0.5 | 0.45 | 0.5 | 0.45 | 0.5 | 0.5 |
| Cab-O-Sil M5P | 0.5 | 0.5 | 0.5 | 0.5 | 0.5 | 0.5 | 0.5 | 0.45 | 0.5 | 0.45 | 0.5 | 0.5 |
| Total | 100 | 100 | 100 | 100 | 100 | 100 | 100 | 90 | 100 | 90 | 100 | 100 |
| Isradipine, USP | | | | | | | | | | | 0.93 | 1.0 |
| Opadry AMB 80W93118, | | | | | | | | 5.4 | | 5.4 | | 6.0 |
| Orange | 000.0 | 000.0 | 000.0 | 000.0 | 000.0 | 000.0 | | 00.00 | | 00.00 | | 000.0 |
| Total | 100 | 100 | 100 | 100 | 100 | 100 | | 95.4 | | 95.4 | | 107 |

1.2. 6 mg Sustained Release Israpidine Tablet

In certain 6 mg tablet embodiments of the present invention, isradipine is combined with the sustained release polymer (e.g., Eudragit® RSPO) from about 13 mg/tablet to about 29 mg/tablet, preferably from about 18 mg/tablet to about 27 mg/tablet, and most preferably about 24.2 mg/tablet. The filler (e.g., 316 Fast Flo®) is present in an amount from about 65 mg/tablet to about 85 mg/tablet, preferably from about 70 mg/tablet to about 80 mg/tablet, and more preferably from about 75 mg/tablet to about 79 mg/tablet. Optionally, magnesium stearate is present in the amount from about 0.45 mg/tablet to about 0.65 mg/tablet, preferably from about 0.5 mg/tablet to about 0.6 mg/tablet, and more preferably about 0.55 mg/tablet. Cab-O-Sil® M5P is optionally present in the amount from about 0.45 mg/tablet to about 0.65 mg/tablet, preferably from about 0.5 mg/tablet to about 0.6 mg/tablet, and more preferably about 0.55 mg/tablet. An exemplary formulation for a 6 mg/tablet of sustained release isradipine is set forth in Table 2.

TABLE 2

Strength (mg)
6

| Ingredient | % | mg/tab |
|---|---|---|
| Isradipine, USP | 5.45 | 6.0 |
| Eudragit RS PO | 22.0 | 24.2 |
| Modified, 316 Fast Flo | 71.55 | 78.7 |
| Magnesium Stearate, NF | 0.5 | 0.55 |
| Cab-O-Sil M5P | 0.5 | 0.55 |
| Total | 100 | 110 |

1.3. 10 mg Sustained Release Israpidine Tablet

In certain 10 mg/tablet embodiments of the present invention, a 10 mg/tablet dose of isradipine is combined with a sustained release polymer (e.g., Eudragit® RSPO) from about 24 mg/tablet to about 56 mg/tablet, preferably from about 32 mg/tablet to about 48 mg/tablet, and most preferably about 40 mg/tablet. A filler (e.g., 316 Fast Flo®) is present in an amount from about 120 mg/tablet to about 160 mg/tablet, preferably from about 130 mg/tablet to about 150 mg/tablet, and more preferably from about 135 mg/tablet to about 145 mg/tablet. Sodium lauryl sulfate is optionally present in the amount from about 0.2 mg/tablet to about 0.6 mg/tablet, preferably from about 0.3 mg/tablet to about 0.46 mg/tablet, and more preferably about 0.4 mg/tablet. Optionally, magnesium stearate is present in the amount from about 0.95 mg/tablet to about 1.05 mg/tablet, preferably from about 0.98 mg/tablet to about 1.02 mg/tablet, and more preferably about 1.0 mg/tablet. Optionally, Cab-O-Sil® M5P is present in the amount from about 0.95 mg/tablet to about 1.05 mg/tablet, preferably from about 0.98 mg/tablet to about 1.02 mg/tablet, and more preferably about 1.0 mg mg/tablet. Opadry® is optionally present in the amount from about 9.6 mg/tablet to about 12 mg/tablet, preferably from about 10.4 mg/tablet to about 11.2 mg/tablet, and more preferably about 10.8 mg mg/tablet. An exemplary formulation for a 10 mg tablet of sustained release isradipine is set forth below in Table 3.

TABLE 3

Strength (mg)
10

| Ingredient | % | mg/tab |
|---|---|---|
| Isradipine, USP | 5.0 | 10 |
| Eudragit RS PO | 22.0 | 44 |
| Modified, 316 Fast Flo | 72.0 | 144 |
| Magnesium Stearate, NF | 0.5 | 1 |
| Cab-O-Sil M5P | 0.5 | 1 |
| Total | 100 | 200 |

1.4. 12 mg Sustained Release Israpidine Tablet

In certain 12 mg tablet embodiments of the present invention, isradipine is combined with the sustained release polymer (e.g., Eudragit® RSPO) from about 30 mg/tablet to about 60 mg/tablet, preferably from about 40 mg/tablet to about 50 mg/tablet, and most preferably about 48.4 mg/tablet. The filler (e.g., 316 Fast Flo®) is present in an amount from about 130 mg/tablet to about 170 mg/tablet, preferably from about 140 mg/tablet to about 160 mg/tablet, and more preferably from about 150 mg/tablet to about 158 mg/tablet. Magnesium stearate is optionally present in the amount from about 1.0 mg/tablet to about 1.2 mg/tablet, preferably from about 1.05 mg/tablet to about 1.15 mg/tablet, and more preferably about 1.1 mg/tablet. Cab-O-Sil® M5P is optionally present in the amount from about 1.0 mg/tablet to about 1.2 mg/tablet, preferably from about 1.05 mg/tablet to about 1.15 mg/tablet, and more preferably about 1.1 mg/tablet. An exemplary formulation for a 12 mg/tablet of sustained release isradipine is set forth in Table 4.

TABLE 4

Strength (mg)
12

| Ingredient | % | mg/tab |
|---|---|---|
| Isradipine, USP | 5.45 | 12.0 |
| Eudragit RS PO | 22.0 | 48.4 |
| Modified, 316 Fast Flo | 71.55 | 157.4 |
| Magnesium Stearate, NF | 0.5 | 1.1 |
| Cab-O-Sil M5P | 0.5 | 1.1 |
| Total | 100 | 220 |

1.5. 18 mg Sustained Release Israpidine Tablet

In certain 18 mg tablet embodiments of the present invention, isradipine is combined with the sustained release polymer (e.g., Eudragit® RSPO) from about 50 mg/tablet to about 90 mg/tablet, preferably from about 60 mg/tablet to about 80 mg/tablet, and most preferably about 72.6 mg/tablet. The filler (e.g., 316 Fast Flo®) is present in an amount from about 200 mg/tablet to about 260 mg/tablet, preferably from about 215 mg/tablet to about 245 mg/tablet, and more preferably from about 230 mg/tablet to about 240 mg/tablet. Magnesium stearate is optionally present in the amount from about 1.55 mg/tablet to about 1.75 mg/tablet, preferably from about 1.6 mg/tablet to about 1.7 mg/tablet, and more preferably about 1.65 mg/tablet. Cab-O-Sil® M5P is optionally present in the amount from about 1.55 mg/tablet to about 1.75 mg/tablet, preferably from about 1.6 mg/tablet to about 1.7 mg/tablet, and more preferably about 1.65 mg/tablet. An exemplary formulation for a 18 mg tablet of sustained release isradipine is set forth in Table 5.

TABLE 5

Strength (mg)
18

| Ingredient | % | mg/tab |
|---|---|---|
| Isradipine, USP | 5.45 | 18.0 |
| Eudragit RS PO | 22.0 | 72.6 |
| Modified, 316 Fast Flo | 71.55 | 236.1 |
| Magnesium Stearate, NF | 0.5 | 1.65 |
| Cab-O-Sil M5P | 0.5 | 1.65 |
| Total | 100 | 330 |

2. Dissolution of Sustained Release Israpidine Tablets

Embodiments of the present invention display a constant and steady rate of dissolution. The dissolution profile, over prolonged period of time, e.g., 24 hours, exhibits a zero order rate of dissolution, or a substantially zero order rate of dissolution. For the purposes herein, “substantially zero-order” means within 20% of zero-order, and preferably within 10% of zero-order, for at least six continuous hours while the israpidine is being delivered, preferably for at least eight continuous hours, more preferably for at least ten continuous hours, and most preferably for at least twelve continuous hours. In preferred variations of the present invention, the release rate of isradipine is controlled by the sustained release polymer, (e.g., Eudragit® RSPO), tablet/capsule size, and the amount of filler, such as lactose.

The dissolution specifications provided herein were obtained under the following conditions:

| | |
|---|---|
| Medium: | 0.2% DDAO |
| Volume: | 500 mL |
| Apparatus: | USP Apparatus 2 (paddles) |
| Speed: | 50 rpm |
| Temperature: | 37° C. +/− 0.5° C. |
| Single Time Point: | 16 hours |
| Profile Time Points: | 0.5, 1, 2, 4, 8, 12, 16, and 24 hours |
| Sample Volume: | 1.5 mL |

In some embodiments, the present invention is directed to a delivery system of sustained release isradipine, wherein a therapeutically effective amount of isradipine in a zero-order sustained release delivery system releases: about 0.5% or more of isradipine at 0.5 hours, preferably about 1% or more; from about 10-40% isradipine at 6 hours, preferably from about 10-35%, and more preferably from about 15-35%; and from about 30%-65% isradipine at 12 hours, preferably from about 40-60% isradipine, and more preferably from about 45-55%.

Optionally, the sustained release delivery system may release from about 60-90% isradipine at 18 hours, preferably about 65-85%, and more preferably about 70-80%; and in some embodiments, at least about 90% isradipine at 24 hours, preferably at least about 95%. In some variations, the delivery system releases about 2% or more of isradipine at 1 hour, preferably about 3% or more, and more preferably about 4% or more.

According to one embodiment, the sustained release delivery system has a 16-hour cumulative release of israpidine of at least 85%. Preferably, the 16-hour cumulative release of israpidine at least 87.5%. More preferably, the 16-hour cumulative release of israpidine is at least 90%. Most preferably, the 16-hour cumulative release of israpidine is at least 92.5%.

2.1 Dissolution of 5 mg Sustained Release Israpidine Tablet

In a 5 mg tablet embodiment, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 3% or more; from about 15-45% isradipine at 6 hours, preferably from about 20-40%, and more preferably from about 25-40%; and from about 50-80% isradipine at 12 hours, preferably from about 65-75% isradipine, and more preferably from about 68-72%.

In a preferred embodiment, the delivery system releases from about 75-95% isradipine at 18 hours, preferably about 80-94%, and more preferably about 85-92%; and at least about 90% isradipine at 24 hours; preferably at least about 95%. In some variations, the delivery system releases about 2% of isradipine at 1 hour, preferably about 4%, and more preferably about 5%.

2.2 Dissolution of 6 mg Sustained Release Israpidine Tablets

### 2.2.1 Embodiment 1

In embodiment 1, the present invention is directed to a delivery system of sustained release isradipine, wherein a therapeutically effective amount of isradipine in a zero-order sustained release delivery system releases: about 0.5% or more of isradipine at 0.5 hours, preferably about 1% or more, and more preferably about 2% or more from about 15-45% isradipine at 6 hours, preferably from about 20-40%, and more preferably from about 25-35%; and from about 45-75% or more isradipine at 12 hours, preferably from about 50-70% or more isradipine, and more preferably from about 55-65%.

In a preferred embodiment, the delivery system releases from about 70-95% or more isradipine at 18 hours, preferably about 75-94% or more, and more preferably about 80-90% or more; and at least about 90% or more isradipine at 24 hours; preferably at least about 95%. In some variations, the delivery system releases about 1% of isradipine at 1 hour, preferably about 2%, and more preferably about 3%.

### 2.2.2 Embodiment 2

In embodiment 2, the present invention is directed to a delivery system of sustained release isradipine, wherein a therapeutically effective amount of isradipine in a zero-order sustained release delivery system releases: about 2% or more of isradipine at 0.5 hours, preferably about 3% or more, and more preferably about 4% or more; from about 30-60% isradipine at 6 hours, preferably from about 35-55%, and more preferably from about 40-50%; and from about 70-95% isradipine at 12 hours, preferably from about 75-92% isradipine, and more preferably from about 80-90%.

In a preferred embodiment, the delivery system releases from about 80-96% isradipine at 18 hours, preferably about 85-95%, and more preferably about 90-95%; and at least about 90% isradipine at 24 hours; preferably at least about 94%. In some variations, the delivery system releases about 2% of isradipine at 1 hour, preferably about 4%, and more preferably about 6%.

### 2.2.3 Embodiment 3

In embodiment 3, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 3% or more; from about 15-45% isradipine at 6 hours, preferably from about 20-40%, and more preferably from about 30-37%; and from about 50-75% isradipine at 12 hours, preferably from about 65-72% isradipine, and more preferably from about 62-70%.

In a preferred embodiment, the delivery system releases from about 80-95% isradipine at 18 hours, preferably about 85-95%, and more preferably about 90-95%; and at least about 90% isradipine at 24 hours; preferably at least about 95%. In some variations, the delivery system releases about 2% of isradipine at 1 hour, preferably about 4%, and more preferably about 5%.

### 2.2.4 Embodiment 4

In embodiment 4, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 3% or more; from about 15-45% isradipine at 6 hours, preferably from about 20-40%, and more preferably from about 25-37%; and from about 50-75% isradipine at 12 hours, preferably from about 55-70% isradipine, and more preferably from about 60-65%.

In a preferred embodiment, the delivery system releases from about 85-98% isradipine at 18 hours, preferably about 89-97%, and more preferably about 90-95%; and at least about 90% isradipine at 24 hours; preferably at least about 95%. In some variations, the delivery system releases about 2% of isradipine at 1 hour, preferably about 4%, and more preferably about 5%.

2.3 Dissolution of 10 mg Sustained Release Israpidine Tablet

In a 10 mg tablet embodiment, the present invention is directed to a delivery system of sustained release isradipine, wherein a therapeutically effective amount of isradipine in a zero-order sustained release delivery system releases: about 1.0% or more of isradipine at 0.5 hours, preferably about 2.0% or more, and more preferably about 3.0% or more; from about 25-65% isradipine at 6 hours, preferably from about 30-60%, and more preferably from about 40-50%; and from about 35-90% isradipine at 12 hours, preferably from about 50-75% isradipine, and more preferably from about 60-65%.

In a preferred embodiment, the delivery system releases from about 50-90% isradipine at 18 hours, preferably about 65-75%, and more preferably about 67-73%; and at least about 90% isradipine at 24 hours; preferably at least about 94%. In some variations, the delivery system releases about 2% of isradipine at 1 hour, preferably about 4%, and more preferably about 6%.

2.4 Dissolution of 12 mg Sustained Release Israpidine Tablets

### 2.4.1 Embodiment 1

In embodiment 1, the present invention is directed to a delivery system of sustained release isradipine, wherein a therapeutically effective amount of isradipine in a zero-order sustained release delivery system releases: about 0.5% or more of isradipine at 0.5 hours, preferably about 0.75% or more, and more preferably about 1% or more; from about 10-30% isradipine at 6 hours, preferably from about 15-25%, and more preferably from about 19-23%; and from about 37-52% isradipine at 12 hours, preferably from about 40-50% isradipine, and more preferably from about 42-46%.

In a preferred embodiment, the delivery system releases from about 60-80% isradipine at 18 hours, preferably about 65-75%, and more preferably about 67-72%; and at least about 90% isradipine at 24 hours; preferably at least about 94%. In some variations, the delivery system releases about 1% of isradipine at 1 hour, preferably about 1.5%, and more preferably about 2%.

### 2.4.2 Embodiment 2

In embodiment 2, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 3% or more; from about 15-35% isradipine at 6 hours, preferably from about 17-32%, and more preferably from about 20-30%; and from about 40-60% isradipine at 12 hours, preferably from about 45-55% isradipine, and more preferably from about 48-52%.

In a preferred embodiment, the delivery system releases from about 65-85% isradipine at 18 hours, preferably about 70-80%, and more preferably about 72-79%; and at least about 90% isradipine at 24 hours; preferably at least about 93%. In some variations, the delivery system releases about 3% of isradipine at 1 hour, preferably about 5%, and more preferably about 7%.

### 2.4.3 Embodiment 3

In embodiment 3, the present invention is directed to a delivery system of sustained release isradipine, wherein a therapeutically effective amount of isradipine in a zero-order sustained release delivery system releases about 0.5% or more of isradipine at 0.5 hours, preferably about 1% or more, and more preferably about 2% or more; from about 15-35% isradipine at 6 hours, preferably from about 17-32%, and more preferably from about 20-30%; and from about 45-70% isradipine at 12 hours, preferably from about 50-65% isradipine, and more preferably from about 55-60%.

In a preferred embodiment, the delivery system releases from about 70-90% isradipine at 18 hours, preferably about 75-85%, and more preferably about 77-83%; and at least about 85% isradipine at 24 hours; preferably at least about 90%. In some variations, the delivery system releases about 3% of isradipine at 1 hour, preferably about 5%, and more preferably about 6%.

### 2.4.4 Embodiment 4

In embodiment 4, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 4% or more; from about 25-50% isradipine at 6 hours, preferably from about 30-45%, and more preferably from about 37-42%; and from about 60-80% isradipine at 12 hours, preferably from about 65-75% isradipine, and more preferably from about 70-73%.

In a preferred embodiment, the delivery system releases from about 75-95% isradipine at 18 hours, preferably about 80-92%, and more preferably about 85-90%; and at least about 90% isradipine at 24 hours; preferably at least about 96%. In some variations, the delivery system releases about 3% of isradipine at 1 hour, preferably about 5%, and more preferably about 7%.

### 2.4.5 Embodiment 5

In embodiment 5, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 3% or more; from about 20-45% isradipine at 6 hours, preferably from about 25-39%, and more preferably from about 27-35%; and from about 50-72% isradipine at 12 hours, preferably from about 55-70% isradipine, and more preferably from about 60-65%.

In a preferred embodiment, the delivery system releases from about 70-95% isradipine at 18 hours, preferably about 75-92%, and more preferably about 80-90%; and at least about 90% isradipine at 24 hours; preferably at least about 95%. In some variations, the delivery system releases about 3% of isradipine at 1 hour, preferably about 5%, and more about preferably 6%.

### 2.4.6 Embodiment 6

In embodiment 6, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 3% or more; from about 40-60% isradipine at 6 hours, preferably from about 42-58%, and more preferably from about 45-55%; and from about 65-95% isradipine at 12 hours, preferably from about 70-95% isradipine, and more preferably from about 80-90.

In a preferred embodiment, the delivery system releases from about 70-99% isradipine at 18 hours, preferably about 80-98%, and more preferably about 85-97%; and at least about 90% isradipine at 24 hours; preferably at least about 95%. In some variations, the delivery system releases about 3% of isradipine at 1 hour, preferably about 5%, and more preferably about 9%.

### 2.4.7 Embodiment 7

In embodiment 7, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 4% or more; from about 20-50% isradipine at 6 hours, preferably from about 25-48%, and more preferably from about 30-45%; and from about 55-85% isradipine at 12 hours, preferably from about 60-80% isradipine, and more preferably from about 65-75%.

In a preferred embodiment, the delivery system releases from about 75-99% isradipine at 18 hours, preferably about 80-98%, and more preferably about 85-95%; and at least about 90% isradipine at 24 hours; preferably at least about 95%. In some variations, the delivery system releases about 4% of isradipine at 1 hour, preferably about 5%, and more preferably about 7%.

2.5 Dissolution of 18 mg Sustained Release Israpidine Tablets

### 2.5.1 Embodiment 1

In embodiment 1, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 4% or more; from about 20-50% isradipine at 6 hours, preferably from about 25-48%, and more preferably from about 30-45%; and from about 55-85% isradipine at 12 hours, preferably from about 60-80% isradipine, and more preferably from about 65-75%.

In a preferred embodiment, the delivery system releases from about 75-99% isradipine at 18 hours, preferably about 80-98%, and more preferably about 85-95%; and at least about 90% isradipine at 24 hours; preferably at least about 98%. In some variations, the delivery system releases about 1% of isradipine at 1 hour, preferably about 2%, and more preferably about 4%.

### 2.5.2 Embodiment 2

In embodiment 2, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 4% or more; from about 5-35% isradipine at 6 hours, preferably from about 10-30%, and more preferably from about 15-25%; and from about 45-65% isradipine at 12 hours, preferably from about 40-60% isradipine, and more preferably from about 45-55%.

In a preferred embodiment, the delivery system releases from about 57-85% isradipine at 18 hours, preferably about 61-80%, and more preferably about 66-76%; and at least about 90% isradipine at 24 hours; preferably at least about 94%. In some variations, the delivery system releases about 0.5% of isradipine at 1 hour, preferably about 1%, and more preferably about 2%.

### 2.5.3 Embodiment 3

In embodiment 3, the present invention is directed to a delivery system of sustained release isradipine, wherein a therapeutically effective amount of isradipine in a zero-order sustained release delivery system releases: about 0.5% or more of isradipine at 0.5 hours, preferably about 1% or more, and more preferably about 2% or more; from about 20-50% isradipine at 6 hours, preferably from about 25-35%, and more preferably from about 20-30%; and from about 50-80% isradipine at 12 hours, preferably from about 55-75% isradipine, and more preferably from about 60-70%.

In a preferred embodiment, the delivery system releases from about 75-99% isradipine at 18 hours, preferably about 80-98%, and more preferably about 85-97%; and at least about 90% isradipine at 24 hours; preferably at least about 95%. In some variations, the delivery system releases about 1% of isradipine at 1 hour, preferably about 2%, and more preferably about 4%.

### 2.5.4 Embodiment 4

In embodiment 4, the present invention is directed to a delivery system of sustained release isradipine, wherein a therapeutically effective amount of isradipine in a zero-order sustained release delivery system releases: about 1% or more of isradipine at 0.5 hours, preferably about 2% or more, and more preferably about 3% or more; from about 20-50% isradipine at 6 hours, preferably from about 25-45%, and more preferably from about 30-40%; and from about 65-95% isradipine at 12 hours, preferably from about 70-90% isradipine, and more preferably from about 75-85%.

In a preferred embodiment, the delivery system releases from about 75-99% isradipine at 18 hours, preferably about 80-98%, and more preferably about 85-97%; and at least about 90% isradipine at 24 hours; preferably at least about 95%. In some variations, the delivery system releases about 1% of isradipine at 1 hour, preferably about 3%, and more preferably about 5%.

### 2.5.5 Embodiment 5

In embodiment 5, the present invention is directed to a delivery system of sustained release isradipine, wherein a therapeutically effective amount of isradipine in a zero-order sustained release delivery system releases: at least about 0.5% or more of isradipine at 0.5 hours, preferably about 1% or more, and more preferably about 2% or more; from about 20-50% isradipine at 6 hours, preferably from about 25-35%, and more preferably from about 20-30%; and from about 50-80% isradipine at 12 hours, preferably from about 55-75% isradipine, and more preferably from about 60-70%.

In a preferred embodiment, the delivery system releases from about 75-99% isradipine at 18 hours, preferably about 80-98%, and more preferably about 85-97%; and at least about 90% isradipine at 24 hours; preferably at least about 95%. In some variations, the delivery system releases about 1% of isradipine at 1 hour, preferably about 2%, and more preferably about 4%.

2.6 Comparison of Dissolution Data for Various Tablets

Table 6 provides the in vitro dissolution profile for exemplary embodiments of the sustained release tablets of isradipine. Data indicate that most embodiments provide a linear pattern of percent isradipine dissolution over time.

TABLE 6
Isradipine Tablet Dissolution Profile

| | Strength (mg) | | | | | | | | | | |
|---|---|---|---|---|---|---|---|---|---|---|---|
| | 5 | | 6 | 12 | 12 | | | | | | |
| | Dyna | 5 | 6 mg/100 mg | 12 mg/200 mg | | 18 | 18 | 6 | 12 | 10 | 10 |
| Time (hours) | Circ | BXR2 | Tab | Tab | | 18 mg/300 Tab | | 6/120 | 12/240 | 10/200 | 10/100 |
| 0 | 0 | 0 | 0 | 0 | 0 | 0 | 0 | 0 | 0 | 0 | 0 |
| 0.5 | 0 | 3 | 2 | 2 | 1 | 2 | 1 | 4 | 2 | 3 | 1 |
| 1 | 0 | 5 | 3 | 3 | 2 | 4 | 2 | 6 | 3 | 6 | 2 |
| 2 | 2 | 11 | 7 | 7 | 5 | 9 | 4 | 10 | 6 | 13 | 5 |
| 4 | 17 | 22 | 18 | 18 | 12 | 20 | 12 | 28 | 13 | 28 | 12 |
| 6 | 53 | 45 | 39 | 33 | 28 | 57 | 29 | 63 | 36 | 63 | 26 |
| 12 | 83 | 69 | 59 | 50 | 44 | 84 | 48 | 85 | 57 | 89 | 38 |
| 16 | 89 | 91 | 81 | 67 | 60 | 91 | 71 | 91 | 77 | 91 | 50 |
| 24 | 94 | 102 | 101 | 93 | 94 | 93 | 94 | 94 | 90 | 94 | 73 |
| Punches | | ¼″ | ¼″ | 9/32″ | 5/16″ | 5/16″ | ⅜″ | ¼″ | 5/16″ | 5/16″ | ¼″ |
| Hardness | | 9.9 | 9.3 | 14.6 | 14.7 | 12.1 | 13.7 | 9.3 | 16.4 | 9.2 | 9.0 |
| (n = 10) [kp] | | | | | | | | | | | |
| Surface Area | | | | | | | | | | | |
| (mm) | | 99.5 | 100 | 154 | 161 | 202 | 219 | 112.3 | 177 | 162 | 98 |
| Volume (mm) | | 77.6 | 78 | 158 | 157 | 238 | 235 | 98 | 188 | 160 | 75 |
| Surface Area/ | | 19.9 | 16.7 | 12.8 | 13.4 | 11.2 | 12.2 | 18.7 | 14.8 | 16.2 | 9.8 |
| Strength | | | | | | | | | | | |
| Surface Area/ | | 1.28 | 1.28 | 0.97 | 1.03 | 0.85 | 0.93 | 1.15 | 0.94 | 1.01 | 1.31 |
| Volume | | | | | | | | | | | |
| Volume/ | | 15.5 | 13.0 | 13.2 | 13.1 | 13.2 | 13.1 | 16.3 | 15.7 | 16.0 | 7.5 |
| Strength | | | | | | | | | | | |

Strength (mg)

| | 6 | 12 | 12 | 18 | 12 | 12 | mg released | | 6 | 18 | 18 |
|---|---|---|---|---|---|---|---|---|---|---|---|
| Time (hours) | 6/110 | 12/220 | 12/220 | 18/330 | 12/220 | 12/220 | BXR2 | 6/110 | 6/110 | 18/330 | 18/330 |
| 0 | 0 | 0 | 0 | 0 | 0 | 0 | 0 | 0 | 0 | 0 | 0 |
| 0.5 | 3 | 4 | 3 | 2 | 6 | 4 | 0.15 | 0.18 | 3 | 3 | 2 |
| 1 | 5 | 7 | 6 | 4 | 9 | 7 | 0.25 | 0.3 | 5 | 5 | 4 |
| 2 | 11 | 14 | 10 | 8 | 17 | 14 | 0.55 | 0.66 | 11 | 11 | 8 |
| 4 | 21 | 27 | 19 | 16 | 32 | 25 | 1.1 | 1.26 | 20 | 21 | 15 |
| 6 | 41 | 51 | 41 | 36 | 64 | 52 | 2.25 | 2.46 | 40 | 45 | 33 |
| 12 | 65 | 71 | 62 | 63 | 86 | 73 | 3.45 | 3.9 | 62 | 78 | 61 |
| 16 | 93 | 85 | 82 | 91 | 95 | 89 | 4.55 | 5.58 | 89 | 94 | 91 |
| 24 | 100 | 96 | 100 | 97 | 98 | 99 | 5.1 | 6 | 98 | 99 | 96 |
| Punches | ¼″ | ¼″ | 9/32″ | ⅜″ | 9/32″ | 9/32″ | | | ¼″ | 3/18″ | 3/18″ |
| Hardness | 9.7 | 12.0 | 14.3 | 14.2 | 9.6 | 11.8 | | | 9.0 | 11.8 | 15.1 |
| (n = 10) [kp] | | | | | | | | | | | |
| Surface Area | | | | | | | | | | | |
| (mm) | 105 | 161 | 163 | 230 | 167 | 164 | | | 105 | 234 | 230 |
| Volume (mm) | 86 | 175 | 174 | 263 | 181 | 177 | | | 86 | 268 | 261 |
| Surface Area/ | 17.5 | 13.4 | 13.6 | 12.8 | 13.9 | 13.7 | | | 17.5 | 13.0 | 12.8 |
| Strength | | | | | | | | | | | |
| Surface Area/ | 1.22 | 0.92 | 0.94 | 0.87 | 0.92 | 0.93 | | | 1.22 | 0.87 | 0.88 |
| Volume | | | | | | | | | | | |
| Volume/ | 14.3 | 14.6 | 14.5 | 14.6 | 15.1 | 14.8 | | | 14.3 | 14.9 | 14.5 |
| Strength | | | | | | | | | | | |

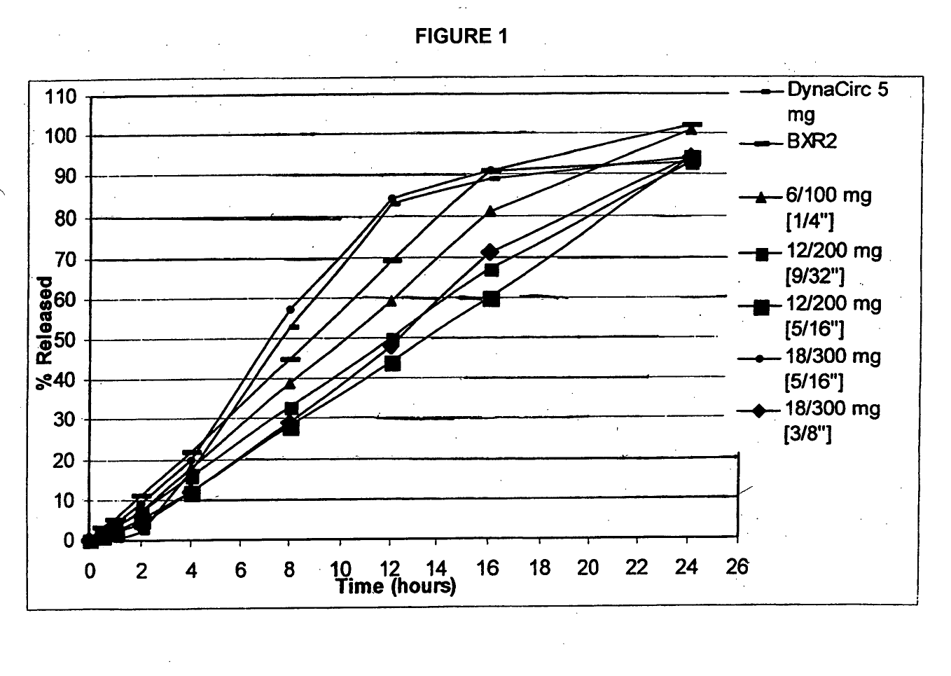

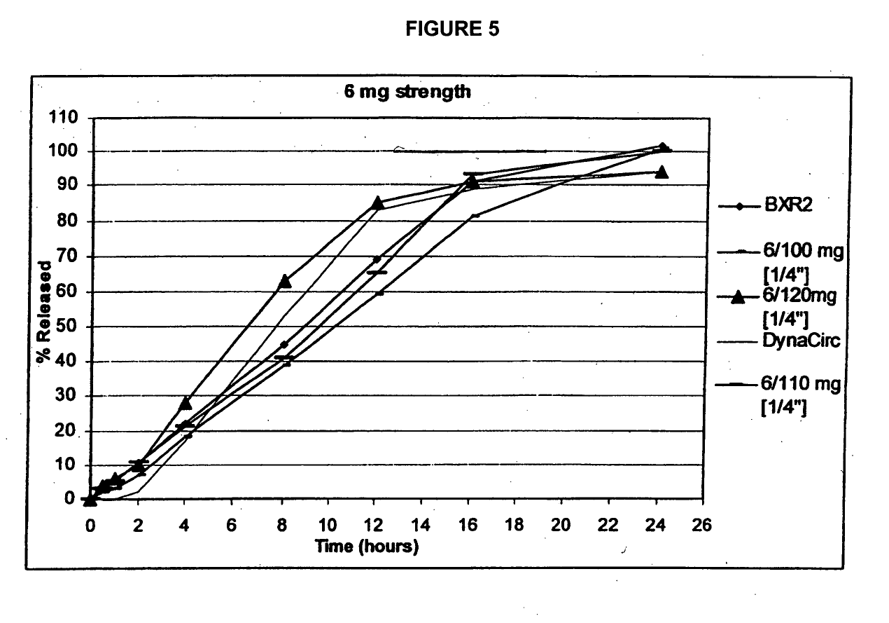

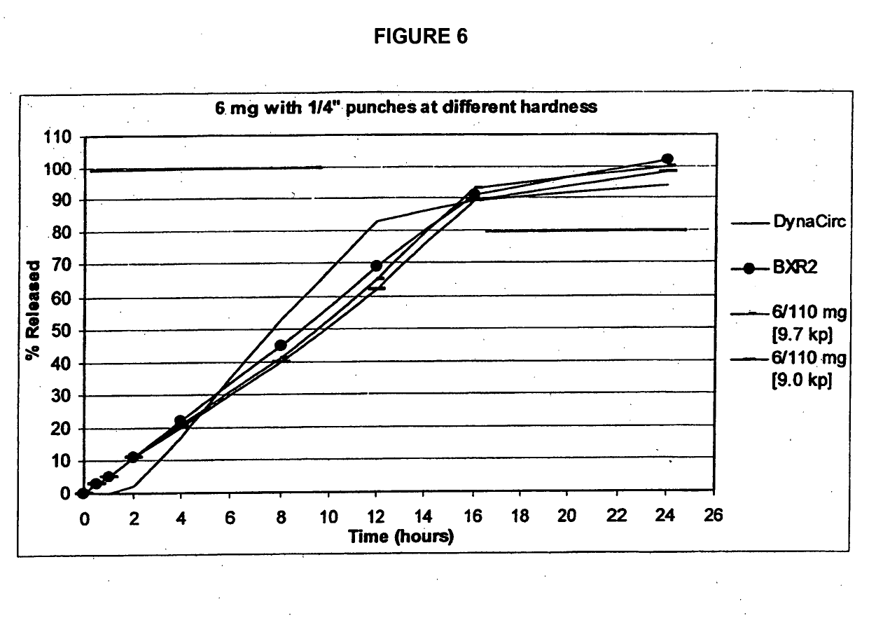

FIGS. 1-10 provide a graphical analysis of the dissolution profile of exemplary formulations of the present invention. In many of the figures, a comparison is shown between the present invention and the conventional formulation of DynaCirc® CR manufactured by Reliant Pharmaceuticals, Liberty Corner, N.J. In contrast to the formulations of the present invention, the conventional formulations of controlled release isradipine exhibit at least one spike in the percent isradipine released at various points in the release chronology. Additionally, as shown in Table 7, the conventional formulation exhibits a delayed release of isradipine.

TABLE 7
DynaCirc 5 and 10 mg Dissolution Data

| | DynaCirc 5 mg | DynaCirc 10 mg |
|---|---|---|
| Time (hours) | % Released | % Released |
| 0 | 0 | 0 |
| 0.5 | 0 | 0 |
| 1 | 0 | 0 |
| 2 | 2 | 1 |
| 4 | 17 | 12 |
| 8 | 53 | 43 |
| 12 | 83 | 73 |
| 16 | 89 | 93 |
| 24 | 94 | 95 |

Collectively, FIGS. 1-10 reflect the substantially zero-order profile associated with the exemplary embodiments of the present invention. In FIG. 1, the embodiments of the present invention display a generally linear percent isradipine dissolution profile over time. In contrast, the conventional formulation of isradipine exhibits a notable spike in percent dissolution and a resulting increased plateau of percent dissolution from about 8 hours to about 18 hours, and also, from about 4 hours to about 12 hours.

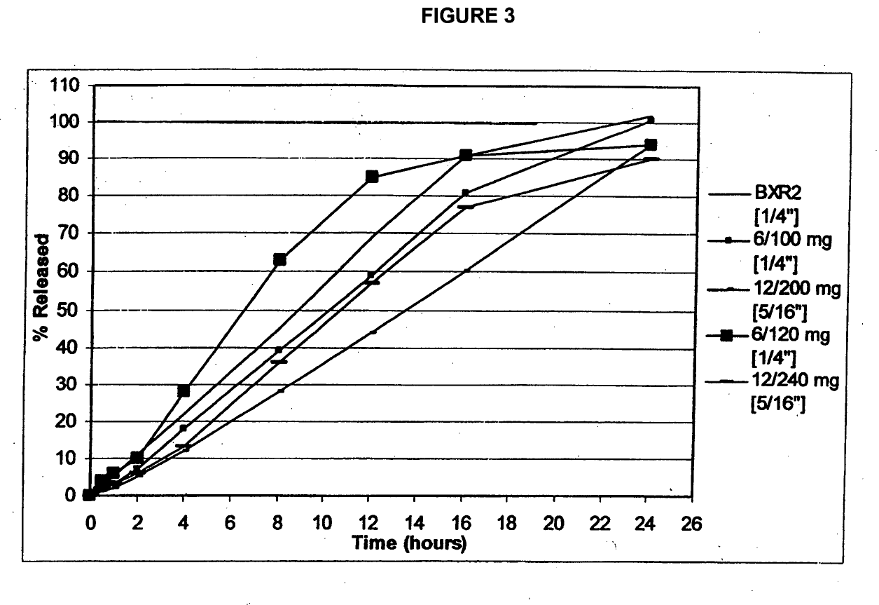

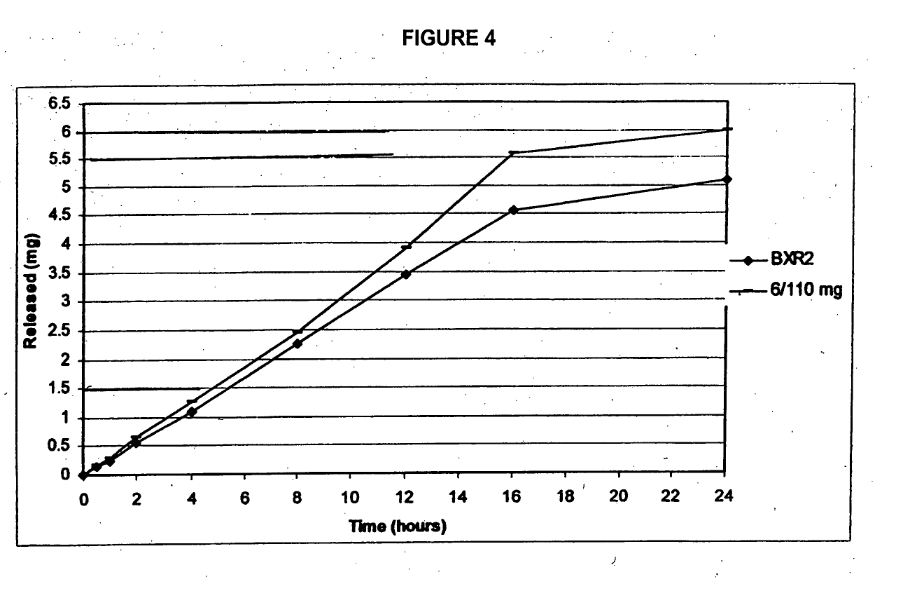

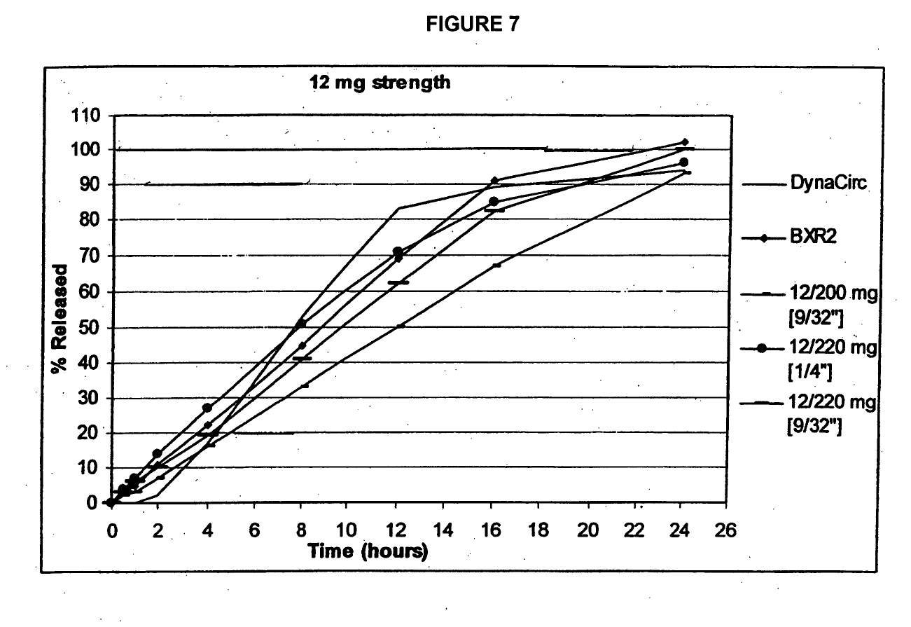

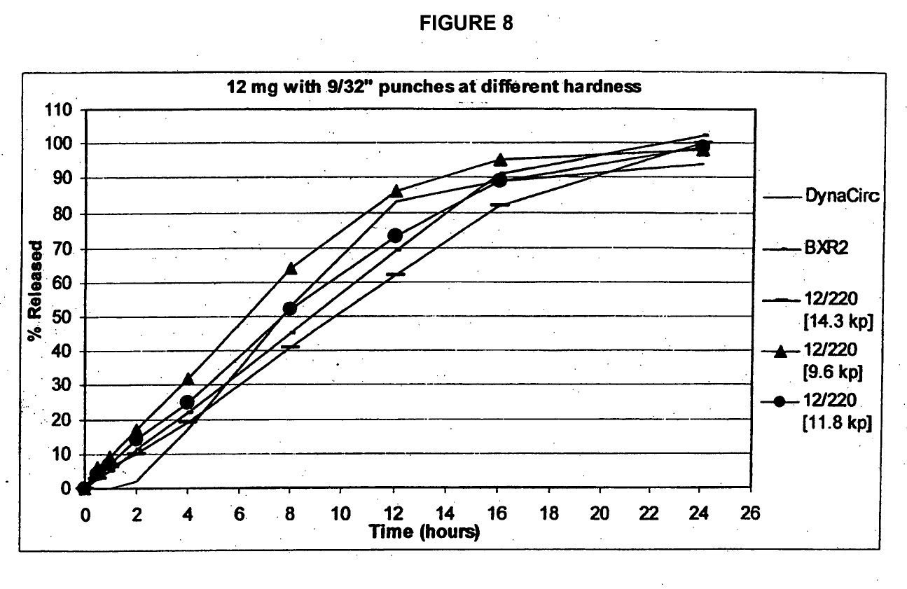

In FIG. 2, the exemplary formulations (5, 6, 12, 18 mg formulations) of the present invention illustrate a steady release of isradipine, unlike the conventional formulation. FIGS. 3-6 illustrate the dissolution of various embodiments of the present invention. In FIG. 4 specifically, both the 5 mg/tablet formulation and the 6 mg/tablet formulation exhibit a generally linear dissolution for approximately 18 hours from dosing. In FIGS. 7 and 8, the 12 mg/tablet exhibits a more linear percent dissolution profile than the conventional tablet. FIG. 8 illustrates a generally linear percent dissolution of 12 mg isradipine tablets with a punch of 9/32″ over a period of 24 hours having various hardness values.

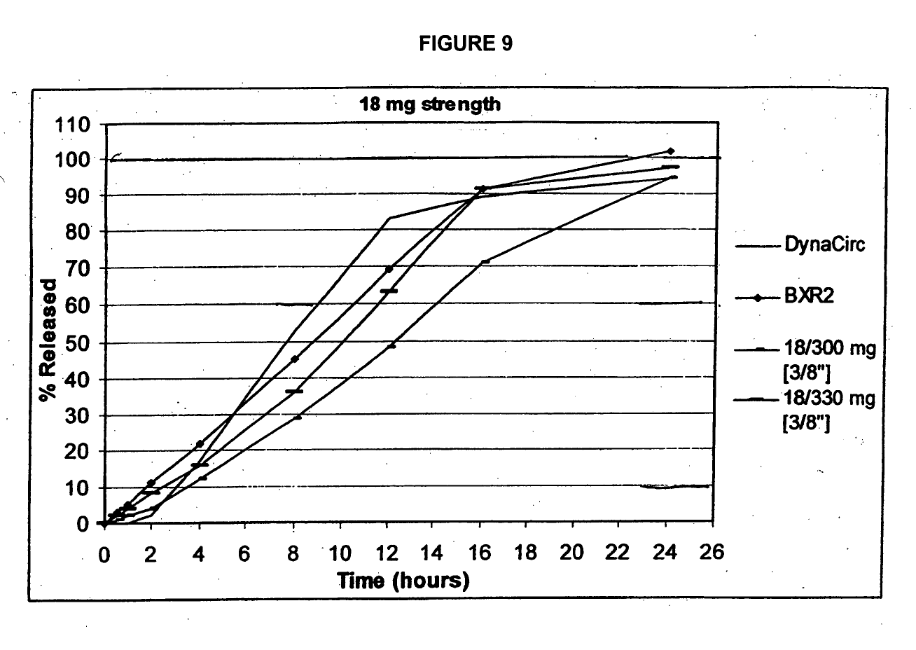

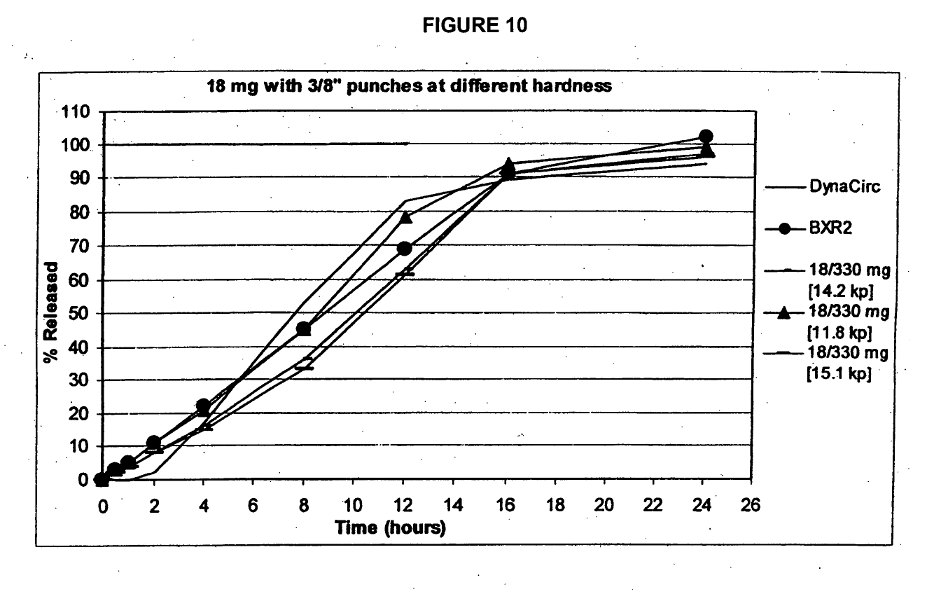

The effect is observed in the exemplary 12 mg/tablets is generally observed in the 18 mg/tablet exemplary embodiments, according to FIGS. 9 and 10. In FIG. 9, the 18 mg/tablet illustrates a more steady release of isradipine than the conventional controlled release tablet. Moreover, in FIG. 10, the 18 mg, ⅜″ punch exemplary embodiments show a substantially steady rate of isradipine release, as compared to the prior art.

Having thus described presently preferred embodiments of the present invention, it will be understood by those skilled in the art that changes in construction and widely differing embodiments and applications of the invention will suggest themselves without departing from the spirit and scope of the present invention. The disclosure and description herein are intended to be illustrative and are not in any sense limiting of the invention.

What is claimed:

1. A sustained release formulation for delivering a therapeutically effective amount of israpidine, said formulation comprising:
a delivery device comprising israpidine and one or more pharmaceutically acceptable excipients; and
an outer coating comprising a sustained release polymer, said outer coating substantially covering said delivery system, wherein said sustained release formulation provides substantially zero-order release of israpidine for at least about six continuous hours while said formulation is being delivered, under the following conditions:

| | |
|---|---|
| Medium: | 0.2% DDAO |
| Volume: | 500 mL |
| Apparatus: | USP Apparatus 2 (paddles) |
| Speed: | 50 rpm |
| Temperature: | 37° C. +/− 0.5° C. |
| Sample Volume: | 1.5 mL. |

2. The sustained release formulation of claim 1, wherein said delivery device is selected from the group consisting of a tablet, tablet cores, and capsule cores.

3. The sustained release formulation of claim 1, wherein said formulation is a tablet comprising:
a 5 mg dose of isradipine,
from about 12 mg/tablet to about 28 mg/tablet of a sustained release polymer, and
wherein said excipient comprises a filler present in an amount of from about 60 mg/tablet to about 80 mg/tablet.

4. The sustained release formulation of claim 1, wherein said formulation comprises a 5 mg dose of isradipine, and
wherein said formulation provides a therapeutically-effective sustained release of said israpidine, whereby about 1% or more of isradipine is released at 0.5 hours; about 15-45% of isradipine is released at 6 hours; and about 50-80% of isradipine is released at 12 hours.

5. The sustained release formulation of claim 4, wherein about 75-95% isradipine is released at 18 hours; and at least about 90% isradipine is released at 24 hours.

6. The sustained release formulation of claim 1, wherein said israpidine is provided in a dosage amount of from about to 2.5 mg to about 25 mg.

7. A method for orally administering a therapeutically effective amount of isradipine, comprising providing the sustained release formulation of claim 1 to a subject.

8. The method of claim 7, wherein a peak plasma level of israpidine is reached about 8-10 hours after administration.

9. The method of claim 7, wherein greater than 50% of the peak plasma concentration is maintained for about 12-24 hours after administration.

10. The method of claim 7, wherein said therapeutically effective amount of sustained release israpidine is for treating heart-related disorders.

11. A sustained release formulation for delivering a therapeutically effective amount of israpidine, said formulation comprising:
a matrix comprising israpidine and a matrix-forming pharmaceutical excipient,
wherein said sustained release formulation provides substantially zero-order release of israpidine for at least about six continuous hours while said formulation is being delivered, under the following conditions:

| | |
|---|---|
| Medium: | 0.2% DDAO |
| Volume: | 500 mL |
| Apparatus: | USP Apparatus 2 (paddles) |
| Speed: | 50 rpm |
| Temperature: | 37° C. +/− 0.5° C. |
| Sample Volume: | 1.5 mL. |

12. The sustained release formulation of claim 11, wherein said formulation is provided as a dosage form selected from the group consisting of tablets and capsules.

13. The sustained release formulation of claim 11, wherein said formulation is a tablet comprising:
a 5 mg dose of isradipine,
from about 12 mg/tablet to about 28 mg/tablet of a sustained release polymer, and
wherein said formulation further comprises a filler present in an amount of from about 60 mg/tablet to about 80 mg/tablet.

14. The sustained release formulation of claim 11, wherein said formulation comprises a 5 mg dose of isradipine, and
wherein said formulation provides a therapeutically-effective sustained release of said israpidine, whereby about 1% or more of isradipine is released at 0.5 hours; about 15-45% of isradipine is released at 6 hours; and about 50-80% of isradipine is released at 12 hours.

15. The sustained release formulation of claim 14, wherein about 75-95% isradipine is released at 18 hours; and at least about 90% isradipine is released at 24 hours.

16. The sustained release formulation of claim 11, wherein said israpidine is provided in a dosage amount of from about to 2.5 mg to about 25 mg.

17. A method for orally administering a therapeutically effective amount of isradipine, comprising providing the sustained release formulation of claim 13 to a subject.

18. The method of claim 17, wherein a peak plasma level of israpidine is reached about 8-10 hours after administration.

19. The method of claim 17, wherein greater than 50% of the peak plasma concentration is maintained for about 12-24 hours after administration.

20. The method of claim 17, wherein said therapeutically effective amount of sustained release israpidine is useful for treating heart-related disorders.
